(19) United States
(12) Patent Application Publication
(10) Pub. No.: US 2023/0142491 A1
(43) Pub. Date: May 11, 2023

(54) PROSTHESIS COSMETIC ELEMENT AND METHOD FOR PRODUCING A MOLDED PART

(71) Applicant: OTTO BOCK HEALTHCARE PRODUCTS GMBH, Wien (AT)

(72) Inventors: Sonja WAGNER, Wien (AT); Walter LUNZER, Wien (AT); Alice SCHERB, Stockerau (AT)

(73) Assignee: OTTO BOCK HEALTHCARE PRODUCTS GMBH, Wien (AT)

(21) Appl. No.: 17/916,546

(22) PCT Filed: Mar. 31, 2021
(86) PCT No.: PCT/EP2021/058471

§ 371 Date: Sep. 30, 2022

(30) Foreign Application Priority Data
Apr. 3, 2020 (DE) 10 2020 109 336.5

(51) Int. Cl. A61F 2/60; B32B 1/08; B32B 5/18; B32B 5/02; B32B 5/24; B29C 44/14; B29C 44/58

(52) U.S. Cl. CPC A61F 2/60; B32B 1/08; B32B 5/18; B32B 5/024; B32B 5/026; B32B 5/245; B29C 44/14; B29C 44/583; B32B 2250/02; B32B 2535/00; B29L 2031/7532

(57) ABSTRACT

A prosthesis cosmetic element comprising a main part made of a foam material and a cavity formed in the main part for receiving a prosthesis component, wherein the main part has an outer face facing away from the prosthesis component and an inner face which delimits the cavity, and a textile is secured to the inner face.

### CROSS REFERENCE TO RELATED APPLICATIONS

This is a national phase application of International Application No. PCT/EP2021/058471, filed 31 Mar. 2021, which claims the benefit of German Patent Application No. 10 2020 109 336.5, filed 3 Apr. 2020, the disclosures of which are incorporated herein, in their entireties, by this reference.

### TECHNICAL FIELD

The invention relates to a prosthesis cosmetic with a base body made of a foam material and with a cavity formed in the base body for receiving a prosthesis component, the base body having an outer side facing away from the prosthesis component and an inner side delimiting the cavity. The invention also relates to a method for producing a molded part, in particular a prosthesis cosmetic, from a foam material with a cavity formed in the molded part for receiving a component, in particular a prosthesis component, in which method a tool core is introduced into a mold cavity of a molding tool and the mold cavity is filled with foam.

### BACKGROUND

Prosthesis cosmetics are used to cover a prosthesis component, among other things with the aim of achieving the most natural appearance possible for the replaced limb, and for protecting the prosthesis component from any external influences. In addition, prosthesis cosmetics can protect the surroundings from direct contact with the prosthesis component, by making available a padding for the prosthesis component.

The direct manufacture of individual prosthesis cosmetics in a primary forming method is highly complex, since a mold first has to be produced which is adapted to the particular user. The usual procedure when producing a prosthesis cosmetic for a specific prosthesis component entails adapting a base body, which has a cavity for receiving the prosthesis component, to the particular patient by removal of material. As a rule, it is sought to produce the prosthesis component with a shape that corresponds to the intact side. The base body is generally made of a light, voluminous material, for example a foam material, which is processed by the relevant orthopedic technician. The shape of the outer contour is worked by grinding or by other cutting methods.

Processing the base body made of a foam material using grinding machines can lead to the foam catching in the grinding machine and tearing. This can lead to injuries to the person processing the base body and to destruction of the product. During the foaming process, a closed or almost closed surface, what is called the integral skin, is formed on the surface of the base body. A structural part or component arranged within the cavity generally executes a movement relative to the inner surface, which can lead to development of noise or to abrasion at the surface of the component that is received.

### SUMMARY

The object of the present invention is to make available a prosthesis cosmetic and a method for producing a molded body from a foam material, with which the disadvantages of the prior art are avoided.

According to the invention, this object is achieved by a prosthesis cosmetic having the features of the main claim and by a method having the features of the additional independent claim. Advantageous embodiments and developments of the invention are disclosed in the subclaims, the description and the figures.

In the prosthesis cosmetic with a base body made of a foam material and with a cavity formed in the base body for receiving a prosthesis component, the base body having an outer side facing away from the prosthesis component and an inner side delimiting the cavity, provision is made that a textile is secured to the inner side of the base body. The dimensional stability of the entire prosthesis cosmetic is improved by the textile, in particular the foamed textile, secured to the inner side of the base body, which textile in one embodiment lines the cavity at least in regions.

In addition, the textile protects against unwanted removal of material during the processing of the base body, in particular if the base body has to be ground down to a small material thickness. The textile also improves work safety during the production of prosthesis cosmetics and ensures that there are fewer rejects. The textile also serves as a reinforcement, which greatly increases the durability of the prosthesis cosmetic. The finished molded body is more durable and no longer tears so easily when subjected to tensile loads. In addition, the arrangement of a textile on the inner side results in less noise being generated during the use of the prosthesis cosmetic, since the integral skin which is otherwise present at these points, and which is created on the inner surface or inner side of the base body on account of the temperature difference between the tool and the foam material introduced, is replaced.

The entire inner side of the cavity can be covered with a textile, as a result of which the dimensional stability of the prosthesis cosmetic is increased overall and the processability of the base body can be made easier when forming the desired outer contour.

In a further development of the invention, the textile is foamed into the base body and is thus an integral part of the base body. It is thereby possible to position the textile in conjunction with the formation of the cavity and to integrate it into the base body without a further work step during the foaming of the mold. The textile or the textile surface is directly back-molded or overmolded with the foam such that a textile inner coating is created in the three-dimensional cavity. The textile is applied to the tool and mechanically anchored in the foaming process. Subsequent gluing-in of a textile is therefore avoided, as also is the formation of the integral skin on the inner side. By virtue of the integral manufacture of the textile reinforcement together with the formation of the cavity, it is possible to arrange the textile over the entire surface area on the inner side, even with complex three-dimensional shapes of the cavity. The textile follows the outer contour of the tool core almost perfectly on account of the pressure that is exerted on the tool core by the foam material during the foaming, which is possible only with simple geometries when the textile is subsequently glued on. In addition, the manufacture of the base body is made easier, since the tool core can be molded directly from the component to be received, or from the prosthesis component to be received, without dimensional allowance.

The textile can be designed as a tube, wherein the tube can be designed in one piece with a closed cross section. In a variant, the tube is produced from a flat basic blank, for example by sewing side edges facing away from each other, after they have been placed on each other or over each other. Other connection techniques for forming a tube or a tubular molded body are also possible, for example gluing or welding. At one end, the tube can have an insertion opening for a tool core which forms the cavity in the base body. The tube is pulled onto the tool core before the mold is filled with foam, such that a textile reinforcement is created which at the same time provides a textile surface structure on the inner side of the base body that is different from an otherwise smooth integral skin surface.

The textile can be flexible, in particular elastic, in order to be able to adapt to the outer contour of a tool core and thus to the sometimes complex inner contour of the cavity. The textile is preferably tear-resistant in order to provide sufficient additional stability of the base body.

The foam material, at least in some regions, cannot penetrate the textile, such that no foam material forms the inner surface of the cavity. A textile inner surface is thus formed.

In the method for producing a molded part, in particular a prosthesis cosmetic, from a foam material with a cavity formed in the molded part for receiving a further component, for example a prosthesis component, in which method a tool core is introduced into a mold cavity of a molding tool and the mold cavity is filled with the foam material, provision is made that the tool core is at least partially covered on its outer side with a textile before the mold cavity is filled with the foam material. The textile can be placed on the tool core. It is likewise possible for the textile to be wrapped around the tool core. The tool core can be completely covered with the textile on its circumference. There is also the possibility of arranging several textiles on the tool core, for example also arranging several layers of a textile or of different textiles on top of one another in order to connect them to the foam material during foaming and to integrate them into the base body. If the tool core protrudes into the mold cavity, the tip protruding into the mold cavity can also be completely covered by the textile. If the tool core protrudes right through the mold cavity, the respective end faces of the tool core are advantageously not covered, and only the lateral surface of the tool core is covered. The textile can also be pulled onto the tool core if it has been manufactured from a tube in a previous work step or has been produced to form a tubular molded body.

A textile tube can be pulled onto the tool core, onto which textile tube the outer shape of the base body is then foamed. After the mold cavity has been filled with foam, the molded body is removed from the mold and the tool core is removed to form the hollow body. The outer contour of the molded body is then generated using at least one cutting method, in particular grinding.

The integral production of the cavity with the textile reinforcement facilitates the production of the base body and the further processing to the final product, or to an intermediate product which is then further processed. The work safety when processing the base body is improved. The durability of the products produced by the foaming is increased, and the usage behavior or operating behavior of the molded bodies is also improved. The foam material can be designed to be stable under pressure, and a certain flexibility and elasticity can also be provided by the foam material.

### BRIEF DESCRIPTION OF THE DRAWINGS

An exemplary embodiment of the invention is explained in more detail below with reference to the accompanying figures, in which:

FIG. 1 shows a schematic flow chart of the production method;

FIG. 2 shows a sewn textile tube and a tool core; and

FIG. 3 shows a partial view of a produced prosthesis cosmetic with a textile reinforcement.

### DETAILED DESCRIPTION

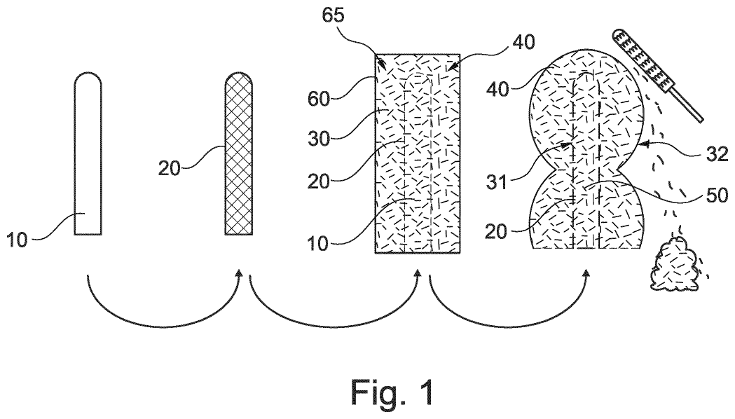

FIG. 1 shows a schematic flow chart for the production of a molded part 30, in particular a prosthesis cosmetic. First, a tool core 10 is manufactured or made available, which tool core 10 is designed to receive a further component, for example a prosthesis component. The outer contour of the tool core 10 can correspond substantially to the outer contour of the component to be received, in particular the prosthesis cosmetic, optionally with additions or deductions and also with formations and projections for securing to the component or the prosthesis cosmetic. A textile 20 is applied to the outer side of the tool core 10. The textile 20 in the exemplary embodiment shown is tubular and designed to be closed at the upper end. It is possible to insert the tool core 10 into the textile 20 through the lower insertion opening. The textile 20 can be formed in one piece, for example knitted or woven, or alternatively it can be made from a flat blank which is sewn or glued accordingly. It is likewise possible to obtain the desired covering from a tubular base body by closing an opening. The textile 20 surrounds the tool core 10 on the outer circumference and at the upper end; the lower end can be left free.

After the tool core 10 has been inserted into the textile 20 or after the tool core 10 has been enveloped with the textile 20 in another way, for example by wrapping, the tool core 10 is inserted into a molding tool 60 that has a mold cavity 65. One end of the tool core 10 with the textile 20 forms a closure with a wall of the molding tool 60; alternatively, one end of the tool core 10 protrudes from the molding tool 60. Alternatively, it is possible that the tool core 10 also extends right through the molding tool 60.

The mold cavity 65 of the molding tool 60 is then filled with a foam material 40, such that a base body 30 is formed from a foam material 40, with a corresponding cavity in its interior after the base body 30 has been removed from the molding tool 60 and the tool core 10 has been removed. The outer contour of the base body 30 corresponds to the inner contour of the molding tool 40 and can be cylindrical, for example. Alternative outer contours are likewise possible, for example conical or roughly approximating to an outer contour of the desired insert shape. The molded body can be used, for example, as a prosthesis cosmetic and can cover a lower leg component of a prosthetic fitting of a lower extremity. A contour of the base body 30, designed approximating to an outer contour of a lower leg, then facilitates the individualization and adaptation to the respective user.

After the removal of the base body 30 and of the tool core 10, the outer contour of the base body 30 is machined, which can be seen in the illustration on the right in FIG. 1. The base body 30 is given the desired contour on the outer side 32 by suitable machining methods, for example by grinding or another cutting method. For this purpose, the foam material 40 is either fastened to a carrier or held by a person. Within the foam material 40, which can also have a certain elasticity, a cavity 50 has been created by the removal of the tool core 10, such that an inner side 31 with an inner surface is present within the base body 30. After the tool core 10 has been removed, the textile 20 is still arranged on the inner side 31 and is permanently connected to the base body 30 and thus to the foam material by the foaming. The textile 20 is foamed into the base body 30 and increases the stability, in particular when the foam material 40 is removed with a corresponding tool, for example with a grinding wheel or a milling head, until very close to the cavity 50. The textile 20 can be designed to be flexible, in particular elastic. The process control during foaming can be chosen in such a way that the foam material 40 does not penetrate through the textile 20, or at least in some regions does not penetrate through the textile 20. This ensures that the inner side 31 of the base body 30 and thus the cavity 50 are covered with the textile 20, at least in those regions where the foam material 40 has not penetrated the textile 20. Even if the foam material 40 penetrates the textile 20, the textile 20 increases the stability within the base body 30 and reduces the tendency of the foam material 40 to tear at low material thicknesses and thereby render the molded body or the prosthesis cosmetic unusable.

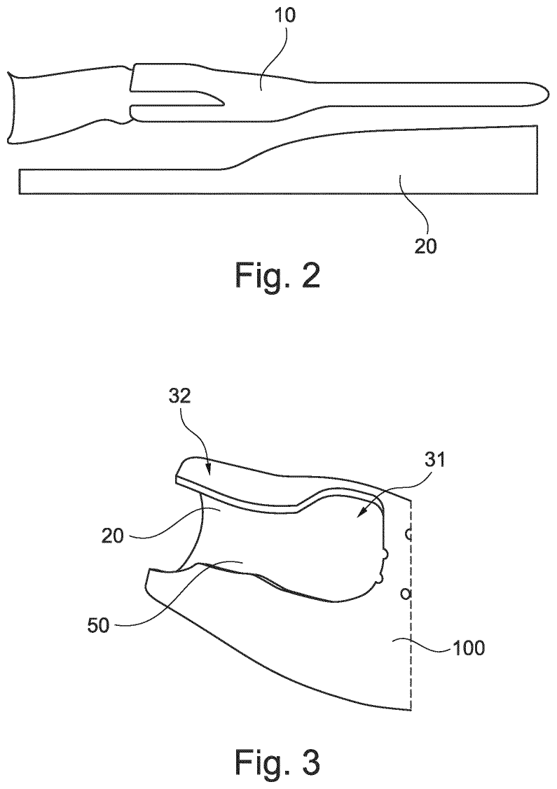

An exemplary embodiment of a tool core 10 and of a textile 20 is shown in FIG. 2. In its left-hand region, the tool core 10 has a thickening with an outer contour which corresponds to a lower part of a prosthetic knee joint. Damper devices, actuators, control devices and the like can be arranged on this lower part. The tool core 10 only reproduces the outer contour of the prosthesis component together with a lower-leg tube connected thereto. The tool core 10 can be made of a plastic or a metal.

The textile 20 that is to be pulled over the tool core 10 is shown below the tool core 10. The textile 20 is produced from a basic blank which is folded along an axis of symmetry and sewn along an outer edge, which is arranged at the top in the exemplary embodiment shown, to form a tubular cover. The textile 20 of FIG. 2 is designed to be open at both ends, and the right-hand end is first pulled over the left-hand end of the tool core 10 until it envelops the outer side of the tool core 10 except for the end faces. The textile 20 is advantageously designed to be flexible, such that it lies tightly against the tool core 10 when inserted into the molding tool 60.

FIG. 3 shows a detailed view of a finished prosthesis cosmetic 100. The outer contour of the prosthesis cosmetic 100 is designed to approximate to the contour of a calf or of a lower leg. The foam material is ground on the outer side 32, and the inner side 31 is lined with the textile 20, such that the cavity 50 is completely provided with a textile 20 on the inner side 31. The initially closed base body has been opened in the region of the calf, opposite the shinbone, i.e. on the rear side, such that the prosthesis cosmetic 100 can be bent open along the separating edge or cutting line and laid around the prosthesis component, which is not shown.

1. A prosthesis cosmetic with a base body made of a foam material and with a cavity formed in the base body for receiving a prosthesis component, the base body having an outer side facing away from the prosthesis component and an inner side delimiting the cavity, wherein a textile is secured to the inner side.

2. The prosthesis cosmetic of claim 1, wherein the entire inner side is covered with a textile.

3. The prosthesis cosmetic of claim 1, wherein the textile is foamed into the base body.

4. The prosthesis cosmetic of claim 1, wherein the textile is designed as a tube.

5. The prosthesis cosmetic of claim 1, wherein the textile is flexible, and/or in particular elastic.

6. The prosthesis cosmetic of claim 1, wherein the foam material, at least in some regions, does not penetrate the textile.

7. A method for producing a molded part from a foam material with a cavity formed in the molded part for receiving a component, in which method a tool core is introduced into a mold cavity of a molding tool and the mold cavity is filled with foam, wherein the tool core is at least partially covered on its outer side with a textile before the mold cavity is filled with the foam material.

8. The method of claim 7, wherein the tool core is completely covered on its circumference with a textile.

9. The method of claim 7, wherein a textile tube is pulled onto the tool core.

10. The method of claim 7, wherein the textile is foamed into the foam material.

11. The method of claim 7, wherein, after the foaming, the molded body is removed from the mold, the tool core is removed to form the cavity, and the outer contour of the molded body is generated using at least one cutting method.

12. A prosthesis cosmetic comprising:
a base body made of a foam material, the base body further including:
a cavity formed in the base body for receiving a prosthesis component;
an outer side which faces away from the prosthesis component; and
an inner side delimiting the cavity;
wherein a flexible textile is secured to at least a portion of the inner side of the base body.

13. The prosthesis cosmetic of claim 12, wherein the entire inner side of the base body is covered with the textile.

14. The prosthesis cosmetic of claim 12, wherein the textile is foamed into the base body.

15. The prosthesis cosmetic of claim 12, wherein the textile is configured as a tube.

16. The prosthesis cosmetic of claim 12, wherein the textile is elastic.

17. The prosthesis cosmetic of claim 12, wherein the foam material does not penetrate the textile.

18. The prosthesis cosmetic of claim 12, wherein the foam material penetrates the textile in at least one region.
